(19) United States
(12) Patent Application Publication
(10) Pub. No.: US 2023/0287512 A1
(43) Pub. Date: Sep. 14, 2023

(54) MARKER FOR RESPONSE TO PD-1/PD-L1 IMMUNOTHERAPY

(71) Applicants: 4C BIOMED LIMITED, London (GB); TEL HASHOMER MEDICAL RESEARCH INFRASTRUCTURE AND SERVICES LTD., Ramat Gan (IL)

(72) Inventors: Eyal GREENBERG, Even Yehuda (IL); Gilli GALORE-HASKEL, Tel Mond (IL); Efrat MERHAVI-SHOHAM, Rehovot (IL); Gal MARKEL, Ramat Gan (IL)

(73) Assignees: 4C BIOMED LIMITED, London (GB); TEL HASHOMER MEDICAL RESEARCH INFRASTRUCTURE AND SERVICES LTD., Ramat Gan (IL)

(21) Appl. No.: 18/130,852

(22) Filed: Apr. 4, 2023

Related U.S. Application Data
(63) Continuation of application No. PCT/IL2021/051196, filed on Oct. 5, 2021.
(60) Provisional application No. 63/182,968, filed on May 2, 2021.
(60) Provisional application No. 63/087,376, filed on Oct. 5, 2020.

(51) Int. Cl. C12Q 1/6886; G01N 33/574

(52) U.S. Cl. CPC C12Q 1/6886; G01N 33/57484; C12Q 2600/158; G01N 2800/52

(57) ABSTRACT

Methods of determining suitability of a subject to be treated with a PD-1/PD-L1 based immunotherapy, comprising receiving a sample from the subject and determining HVEM levels in the sample, wherein expression of HVEM above a predetermined threshold indicates the subject is suitable to be treated by the immunotherapy or of determining suitability of a subject non-responsive to a PD-1/PD-L1 based immunotherapy to be treated by an HVEM based immunotherapy, comprising receiving a sample from the subject and determining T cell levels in the sample, wherein T cell levels above a predetermined threshold indicates the subject is suitable to be treated by the HVEM based immunotherapy are provided.

### CROSS REFERENCE TO RELATED APPLICATIONS

This application is a Bypass Continuation of PCT Patent Application No. PCT/IL2021/051196 having International filing date of Oct. 5, 2021, which claims the benefit of priority of U.S. Provisional Pat. Application No. 63/087,376 filed on Oct. 5, 2020, and U.S. Provisional Pat. Application No. 63/182,968 filed on May 2, 2021, the contents of which are all incorporated herein by reference in their entirety.

### REFERENCE TO AN ELECTRONIC SEQUENCE LISTING

The contents of the electronic sequence listing (4CB-TEL-P-006-US.xml; Size: 23,640 bytes; and Date of Creation: Apr. 4, 2023) is herein incorporated by reference in its entirety.

### FIELD OF INVENTION

The present invention is in the field of immunotherapy diagnostics.

### BACKGROUND OF THE INVENTION

Immunotherapies designed to enhance an immune response are considered activating immunotherapies and are at the forefront of cancer treatment. Currently, immune checkpoint blockade therapy is successfully being used for treatment of advanced non-small cell lung cancers (NSCLC), metastatic melanoma, advanced renal cell carcinoma (RCC), urothelial cancer, MSI-high tumors, Merkel cell carcinoma, squamous cell carcinoma, hepatocellular carcinoma, cervical carcinoma, head and neck cancer, pleural mesothelioma and many others. A few such drugs, developed and manufactured by different companies, have shown great promise in different cancer types and have been FDA approved, including antibodies to the immune checkpoints programmed cell death protein 1 receptor (PD1), programmed cell death protein 1 ligand (PDL1) and cytotoxic T lymphocyte-associated protein 4 (CTLA-4). Patient response rate, however, is still not optimal, as only 10% - 40% of treated patients usually benefit, and at the same time patients may suffer from post immunotherapy treatments side effects. In addition, treatment with these agents, may induce resistance through upregulation of additional immune checkpoints. Combination of anti-PD1 and anti-CTLA-4 therapy in melanoma patients has demonstrated higher response rate (60%) as compared to single agent, however this combination therapy involves also severe treatment-related adverse effects. Thus, there is a clear need for new antitumor immune activating agents.

Herpesvirus entry mediator (HVEM) is a protein found on the surface of various cell types, including hematopoietic and non-hematopoietic cells. HVEM acts as a receptor for canonical TNF-related ligands such as LIGHT and LTα, thus acting as a signaling receptor. However, it also acts as a ligand for immunoglobulin (Ig) superfamily molecules such as inhibitory receptors BTLA and CD160. Therefore, bidirectional signaling is possible for the HVEM-mediated signaling network, which can be involved in positive or negative immunological reactions under different contexts. Dysregulation of this network is involved in the pathogenesis of autoimmune diseases, and inflammatory diseases as well as cancer, making HVEM a target for immunotherapy. Methods of determining subjects that will respond to specific immunotherapies are greatly needed.

### SUMMARY OF THE INVENTION

The present invention provides, in some embodiments, methods of determining suitability of a subject to be treated with PD-1/PD-L1 based immunotherapy, comprising receiving a sample from the subject and determining HVEM levels in the sample, wherein expression of HVEM above a predetermined threshold indicates the subject is suitable to be treated by the immunotherapy and/or methods of determining suitability of a subject non-responsive to treatment by a PD-1/PD-L1 based immunotherapy, to be treated by an HVEM based immunotherapy, comprising receiving a sample from the subject and determining T cell levels in the sample, wherein T cell levels above a predetermined threshold indicates the subject is suitable to be treated by the an HVEM based immunotherapy.

According to a first aspect, there is provided a method of determining suitability of a subject in need thereof to be treated by a PD-1/PD-L1 based immunotherapy, the method comprising receiving a sample from the subject and determining Herpesvirus entry mediator (HVEM) levels in the sample, wherein expression of HVEM above a predetermined threshold indicates the subject is suitable to be treated by the PD-1/PD-L1 based immunotherapy.

According to another aspect, there is provided a method of determining suitability of a subject non-responsive to PD-1/PD-L1 based immunotherapy to be treated with an HVEM based immunotherapy, the method comprising receiving a sample from the subject and determining T cell levels in the sample, wherein T cell levels above a predetermined threshold indicates the subject is suitable to be treated by the HVEM based immunotherapy.

According to some embodiments, the predetermined threshold is an expression level in a healthy sample or in a disease sample from a non-responder to the PD-1/PD-L1 based immunotherapy.

According to some embodiments, the HVEM levels is HVEM mRNA levels or HVEM protein levels.

According to some embodiments, the HVEM levels is HVEM protein levels and the determining comprises contacting the sample with an agent that binds specifically to an extracellular domain of HVEM.

According to some embodiments, the antibody comprises three heavy chain CDRs (CDR-H) and three light chain CDRs (CDR-L), wherein: CDR-H1 comprises the amino acid sequence set forth in SEQ ID NO: 1 (SYAMS), CDR-H2 comprises the amino acid sequence as set forth in SEQ ID NO: 2 (AISGSGGSTYYADSVKG), CDR-H3 comprises the amino acid sequence as set forth in SEQ ID NO: 3 (APGDYTAYFDY), CDR-L1 comprises the amino acid sequence as set forth in SEQ ID NO: 4 (RASQSVSSYLA), CDR-L2 comprises the amino acid sequence as set forth in SEQ ID NO: 5 (GASSRAT), and CDR-L3 comprises the amino acid sequence as set forth in SEQ ID NO: 6 (QQYGSSPPYT).

According to some embodiments, the antibody or antigen binding fragment thereof comprises a detectable moiety or wherein the determining further comprises contacting the sample with a secondary antibody comprising a detectable moiety.

According to some embodiments, the sample is a disease sample.

According to some embodiments, the disease sample is a biopsy.

According to some embodiments, the sample is a bodily fluid.

According to some embodiments, the bodily fluid is blood or plasma.

According to some embodiments, the subject suffers from a disease or condition treatable by PD-1/PD-L1 based immunotherapy.

According to some embodiments, the disease is cancer.

According to some embodiments, the cancer is a solid cancer.

According to some embodiments, detecting HVEM levels comprises detecting HVEM levels on a cell surface and/or in a cytoplasm of a cell in the sample.

According to some embodiments, detecting HVEM levels comprises detecting circulating HVEM levels.

According to some embodiments, the PD-1/PD-L1 based immunotherapy is PD-1 and/or PD-L1 blockade.

According to some embodiments, the method further comprises treating a suitable subject with the PD-1/PD-L1 based immunotherapy or HVEM based immunotherapy.

According to some embodiments, the subject suffers from a disease or condition treatable by PD-1/PD-L1 based immunotherapy and HVEM based immunotherapy.

According to some embodiments, the disease is cancer.

According to some embodiments, the cancer is a solid cancer.

According to some embodiments, the sample is a disease sample.

According to some embodiments, the disease sample is a biopsy.

According to some embodiments, determining T cell levels comprises measuring CD3 expression in the sample.

According to some embodiments, determining T cell levels comprises counting T cells.

According to some embodiments, the T cells are tumor infiltrating lymphocytes (TILs).

According to some embodiments, a suitable subject is suitable to receive a combined HVEM based immunotherapy and a PD-1/PD-L1 based immunotherapy.

According to some embodiments, the PD-1/PD-L1 based immunotherapy is PD-1 and/or PD-L1 blockade, the HVEM based immunotherapy is HVEM blockade, or both.

According to some embodiments, the method of the invention further comprises treating a suitable subject with HVEM based immunotherapy.

According to some embodiments, the method of the invention further comprises treating a suitable subject with the PD-1/PD-L1 based immunotherapy in combination with the HVEM based immunotherapy.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1G. HVEM expression in responders and non-responders to PD-1 blockade: (1A-1D) Micrographs showing CD3, PD-L1 and HVEM immunostaining in (1A) T cells, (1B) melanoma cells, (1C) non-responders and (1D) responders. Arrows indicate particular areas of high expression. (1E-1G) (Top) Charts summarizing expression levels of (1E) CD3, (1F) PD-L1 and (1G) HVEM in responders and non-responders, as well as the statistical significance of the differences in expression. (Left-bottom) Bar graphs of (1E) CD3, (1F) PD-L1 and (1G) HVEM average expression as measured by total positive pixels/field for all images. (Right-bottom) Dot plots of (1E) CD3, (1F) PD-L1 and (1G) HVEM expression per sample as measured by total positive pixels/field.

FIGS. 2A-2B. Scatter plots of the relationship between (2A) CD3 expression and PD-L1 expression in tumor samples from responders (top) and non-responders (bottom) to PD-1/PD-L1 based immunotherapy and (2B) CD3 expression and HVEM expression in tumor samples from responders (top) and non-responders (bottom) to PD-1/PD-L1 based immunotherapy. Correlation coefficient (r) and statistical significance (p) are given for each correlation.

FIGS. 3A-3B. Scatter plots of the relationship between CD3 expression and HVEM expression on (3A) T cells and (3B) tumor cells in samples from non-responders.

### DETAILED DESCRIPTION OF THE INVENTION

The present invention, in some embodiments, provides methods of determining suitability of a subject to be treated with PD-1/PD-L1 based immunotherapy, comprising receiving a sample from the subject and determining HVEM levels in the sample, wherein expression of HVEM above a predetermined threshold indicates the subject is suitable to be treated by the immunotherapy. The present invention, in some embodiments, also provides methods of determining suitability of a subject non-responsive to treatment by a PD-1/PD-L1 based immunotherapy, to be treated by an HVEM based immunotherapy, comprising receiving a sample from the subject and determining T cell levels in the sample, wherein T cell levels above a predetermined threshold indicates the subject is suitable to be treated by the HVEM based immunotherapy.

It is well known that not all subjects respond to PD-1/PD-L1 based immunotherapies. Even some subjects with high expression of PD-1 or a high PD-L1 expressing cancer inexplicably do not respond. This invention is based on the surprising finding that HVEM expression unexpectedly correlates with response to PD-1/PD-L1 based immunotherapies. It has been suggested that HVEM is a negative prognostic marker for cancer therapy, but as is described hereinbelow, it was found that samples from subjects that responded to PD-1 blockade showed significantly higher protein expression of HVEM. This demonstrates that HVEM is indeed a prognostic marker for PD-1/PD-L1 responsiveness. The invention is further based on the surprising finding that among subject who do not respond to PD-1/PD-L1 immunotherapies T cell infiltration into the cancer is indicative of HVEM levels and thus response to an HVEM based immunotherapy or a combined PD-1/PD-L1 and HVEM based immunotherapy treatment.

By a first aspect, there is provided a method of determining suitability of a subject to be treated with a PD-1/PD-L1 based immunotherapy, comprising receiving a sample from the subject and determining HVEM levels in the sample, wherein expression of HVEM above a predetermined threshold indicates the subject is suitable to be treated by the immunotherapy.

By another aspect, there is provided a method of determining suitability of a subject that does not respond to a PD-1/PD-L1 based immunotherapy to be treated with an HVEM based immunotherapy, comprising receiving a sample from the subject and determining T cell levels in the sample, wherein T cell levels above a predetermined threshold indicates the subject is suitable to be treated by the HVEM based immunotherapy.

By another aspect, there is provided a method of determining suitability of a subject to be treated with a combination of a PD-1/PD-L1 based immunotherapy, and an HVEM based immunotherapy, comprising receiving a sample from the subject and determining T cell levels in the sample, wherein T cell levels above a predetermined threshold indicates the subject is suitable to be treated by the combination immunotherapy.

In some embodiments, the method is a method of assessing responsiveness. In some embodiments, assessing comprises predicting. As used herein the term “assessing responsiveness” refers to determining the likelihood that the subject will respond to treatment, namely the success or failure of treatment. In some embodiments, assessing responsiveness comprises characterizing the subject as a responder to treatment. In some embodiments, assessing responsiveness comprises characterizing the subject as a non-responder to treatment.

The term “response” or “responsiveness” to treatment refers to an improvement in at least one relevant clinical parameter as compared to an untreated subject diagnosed with the same pathology (e.g., the same type, stage, degree and/or classification of the pathology), or as compared to the clinical parameters of the same subject prior to treatment. Response need not be complete recovery or eradication of a disease, but rather may be only an improvement in the disease.

The term “non-responder” to treatment refers to a patient not experiencing an improvement in at least one of the clinical parameters and is diagnosed with the same condition as an untreated subject diagnosed with the same pathology (e.g., the same type, stage, degree and/or classification of the pathology), or experiencing the clinical parameters of the same subject prior to treatment. In some embodiments, a non-responder is a subject in which the disease progresses. In some embodiments, a non-responder is a subject in which the disease does not show improvement. In some embodiments, the disease is the disease after treatment.

In some embodiments, the method is a method of determining a non-responder likely to be converted to a responder by administering HVEM based immunotherapy. In some embodiments, the method is a method of determining suitability of a non-responder to be converted to a responder by administering HVEM based immunotherapy. In some embodiments, the method is a method of determining suitability of a non-responder to PD-1/PD-L1 based immunotherapy to receive HVEM based immunotherapy. In some embodiments, T cell levels above a predetermined threshold indicate the subject is suitable to receive HVEM based immunotherapy. In some embodiments, a non-responder is a non-responder to PD-1/PD-L1 based immunotherapy.

In some embodiments, HVEM is mammalian HVEM. In some embodiments, HVEM is human HVEM. In some embodiments, HVEM is any one of mouse, monkey and human HVEM. In some embodiments, HVEM is membrane bound HVEM. In some embodiments, HVEM is HVEM on a cell. In some embodiments, HVEM is HVEM on a cell surface. In some embodiments, HVEM is cytoplasmic HVEM. In some embodiments, HVEM is soluble HVEM. In some embodiments, HVEM is circulating HVEM.

In some embodiments, HVEM is Tumor necrosis factor receptor superfamily member 14 (TNFRSF14). In some embodiments, HVEM is CD270. In some embodiments, HVEM is a receptor of BTLA. In some embodiments, HVEM is a ligand of BTLA. In some embodiments, BTLA is CD272. In some embodiments, HVEM is a receptor of Tumor necrosis factor superfamily member 14 (TNFSF14). In some embodiments, HVEM is a ligand of TNFSF14. In some embodiments, the TNFSF14 is LIGHT. In some embodiments, TNFSF14 is CD258. In some embodiments, HVEM is a receptor of CD160. In some embodiments, HVEM is a ligand of CD160. In some embodiments, HVEM is a receptor of lymphotoxin alpha (LTα). In some embodiments, HVEM is a ligand of LTα. In some embodiments, LTα is TNF-β.

In some embodiments, HVEM levels are HVEM expression. In some embodiments, HVEM is HVEM protein. In some embodiments, HVEM is HVEM mRNA. In some embodiments, HVEM expression is expression on a cell surface. In some embodiments, HVEM expression is circulating expression. In some embodiments, HVEM expression is tumor expression. In some embodiments, HVEM expression is cancer HVEM expression. In some embodiments, HVEM expression is subject expression. In some embodiments, HVEM expression is immune cell HVEM expression. In some embodiments, the immune cell is a tumor immune cell. In some embodiments, a tumor immune cell is a tumor resident immune cell. In some embodiments, the immune cell is a tumor infiltrating lymphocyte (TIL).

In some embodiments, expression is protein expression. In some embodiments, expression is mRNA expression. In some embodiments, expression is gene expression. In some embodiments, the determining comprises contacting the sample with an agent that binds HVEM. In some embodiments, the determining comprises contacting the sample with an agent that binds HVEM mRNA. In some embodiments, the determining comprises contacting the sample with an agent that binds HVEM DNA. In some embodiments, binding comprises specific binding. In some embodiments, the determining comprises contacting the sample with an anti-HVEM antibody. In some embodiments, contacting comprises incubating. In some embodiments, the contacting is under conditions sufficient for binding of the agent to HVEM. In some embodiments, the contacting is in vitro. In some embodiments, the contacting is in vivo. In some embodiments, the conditions are physiological conditions. In some embodiments, the conditions are for a time sufficient for binding. In some embodiments, binding is hybridization. In some embodiments, binding is bonding. Conditions for protein-protein binding (e.g., antibody binding) in samples are well known in the art and a skilled artisan can select the proper conditions for this assay. Similarly, conditions for nucleic acid hybridization are also well known in the art and can be selected by the skilled artisan.

In some embodiments, expression is expression above a predetermined threshold. In some embodiments, the threshold is an expression level in a healthy sample. In some embodiments, the threshold is T cell level in a healthy sample. In some embodiments, the threshold is an expression level in a healthy population. In some embodiments, the threshold is T cell level in a healthy population. In some embodiments, expression level is average expression. In some embodiments, T cell level is the average level. In some embodiments, average is average in sample. In some embodiments, average is average in a defined region. In some embodiments, average is average in a population. In some embodiments, the threshold is an expression level in a disease sample from a non-responder. In some embodiments, the threshold is the average T cell level in disease samples from non-responders. In some embodiments, non-responders are non-responders to combined therapy. In some embodiments, a responder is a responder to PD-1/PD-L1 based immunotherapy. In some embodiments, a non-responder is a non-responder to PD-1/PD-L1 based immunotherapy. In some embodiments, the threshold is an expression level in a non-responding population. In some embodiments, the threshold is a T cell level in a non-responding population. In some embodiments, T cell level is T cell number.

In some embodiments, the subject is a subject in need thereof. In some embodiments, the subject is a mammal. In some embodiments, the subject is a human. In some embodiments, the subject suffers from a disease or condition. In some embodiments, the disease or condition is treatable by PD-1 based immunotherapy. In some embodiments, the disease or condition is treatable by PD-L1 based immunotherapy. In some embodiments, the disease or condition is treatable by PD-1 and/or PD-L1 based immunotherapy. In some embodiments, the disease or condition is treatable by PD-1/PD-L1 based immunotherapy. In some embodiments, the disease is cancer. In some embodiments, the cancer is PD-L1 expressing cancer. In some embodiments, the cancer is a PD-L1 positive cancer. In some embodiments, the disease or condition is treatable by HVEM based immunotherapy. In some embodiments, the cancer is HVEM expressing cancer. In some embodiments, the cancer is an HVEM positive cancer. In some embodiments, the cancer is HVEM and PD-L1 expressing cancer. In some embodiments, the cancer is an HVEM and PD-L1 double positive cancer. In some embodiments, the cancer is a solid cancer. In some embodiments, the cancer is a hematological cancer. In some embodiments, the cancer is skin cancer. In some embodiments, the skin cancer is melanoma. In some embodiments, the cancer is selected from skin cancer, lung cancer, renal cancer, urothelial cancer, hepatic cancer, cervical cancer, head and neck cancer, bladder cancer and high microsatellite instability (MSI) tumors. In some embodiments, the subject is not responsive to PD-1/PD-L1 based immunotherapy. In some embodiments, the subject is not responsive to PD-1 based immunotherapy. In some embodiments, the subject is not responsive to PD-L1 based immunotherapy.

In some embodiments, PD-1/PD-L1 based immunotherapy is PD-1 based immunotherapy and/or PD-L1 based immunotherapy. In some embodiments, PD-1/PD-L1 based immunotherapy is either PD-1 based immunotherapy or PD-L1 based immunotherapy. In some embodiments, PD-1/PD-L1 based immunotherapy is PD-1 based immunotherapy, PD-L1 based immunotherapy or both. In some embodiments, PD-1/PD-L1 based immunotherapy is a combination of PD-1 and PD-L1 based immunotherapy.

In some embodiments, a PD-1/PD-L1 based immunotherapy comprises PD-1 blockade. In some embodiments, a PD-1/PD-L1 based immunotherapy comprises PD-L1 blockade. In some embodiments, a PD-1/PD-L1 based immunotherapy comprises PD-1/PD-L1 blockade. In some embodiments, a PD-1/PD-L1 based immunotherapy comprises an anti-PD-1 blocking antibody. In some embodiments, a PD-1/PD-L1 based immunotherapy comprises an anti-PD-L1 blocking antibody. In some embodiments, the immunotherapy comprises inhibiting interaction between PD-1 and one of its ligands. In some embodiments, a PD-1 ligand is selected from PD-L1 and PD-L2. In some embodiments, the immunotherapy comprises inhibiting PD-1, PD-L1 or PD-L2. In some embodiments, the immunotherapy comprises inhibiting PD-1 to PD-L1 interaction. In some embodiments, the immunotherapy comprises inhibiting PD-1 to PD-L2 interaction. In some embodiments, the method further comprises inhibiting PD-1 to PD-L1 or PD-L2 interaction. In some embodiments, inhibiting interaction comprises administering an agent that inhibits PD-1 to PD-L1 interaction or PD-1 to PD-L2 interaction. In some embodiments, inhibiting interaction comprises administering an agent that inhibits interaction between PD-1 and at least one of its ligands. In some embodiments, at least one ligand is two ligands. In some embodiments, inhibiting interaction comprises PD-1/PD-L1 blockade. In some embodiments, inhibiting interaction comprises PD-1/PD-L1 or PD-L2 blockade. In some embodiments, an agent that inhibits interaction is an anti-PD-1 or anti-PD-L1 antibody. In some embodiments, an agent that inhibits interaction is an anti-PD-1, anti-PD-L1 or anti-PD-L2 antibody. In some embodiments, an agent that inhibits interaction is a PD-1/PD-L1 inhibitor. In some embodiments, an agent that inhibits interaction is a PD-1/PD-L1/PD-L2 inhibitor. In some embodiments, the antibody is a blocking antibody. PD-L1/L2 and PD-1 therapies are well known in the art and include but are not limited to nivolumab (Opdivo), pembrolizumab (Keytruda), atezolizumab, avelumab, durvalumab, cemiplimab (Libtayo), pidilizumab, Dostarlimab, AMP-224, AMP-514 and PDR001. In some embodiments, the anti-PD-1 immunotherapy is selected from Nivolumab or Pembrolizumab.

In some embodiments, a HVEM based immunotherapy comprises HVEM blockade. In some embodiments, a HVEM based immunotherapy comprises an anti-HVEM blocking antibody. In some embodiments, the immunotherapy comprises inhibiting interaction between HVEM and BTLA. In some embodiments, the immunotherapy comprises inhibiting HVEM. In some embodiments, the immunotherapy comprises inhibiting HVEM and BTLA interaction. In some embodiments, the immunotherapy comprises inhibiting HVEM and CD160 interaction. In some embodiments, the immunotherapy does not comprise inhibiting HVEM and TNFSF14 interaction. In some embodiments, the immunotherapy does not comprise inhibiting HVEM and lymphotoxin alpha (LTα) interaction. In some embodiments, inhibiting interaction comprises administering an agent that inhibits interaction. In some embodiments, LTα is TNF-β. In some embodiments, the TNFSF14 is LIGHT. In some embodiments, TNFSF14 is CD258. In some embodiments, an agent that inhibits interaction is an anti-HVEM antibody. In some embodiments, an agent that inhibits interaction is an anti-BTLA antibody. In some embodiments, an agent that inhibits interaction is an HVEM inhibitor. In some embodiments, the antibody is a blocking antibody. HVEM therapies are well known in the art and include but are not limited to blocking antibodies disclosed in International Patent Application WO2020222235 herein incorporated by reference in its entirety.

In some embodiments, the sample is a disease sample. In some embodiments, the sample is a cancer sample. In some embodiments, the sample comprises cells. In some embodiments, the cells are disease cells. In some embodiments, the cells are cancer cells. In some embodiments, the sample comprises tissue. In some embodiments, the sample is a biopsy. In some embodiments, the sample is a bodily fluid. In some embodiments, the bodily fluid is selected from at least one of: blood, serum, plasma, gastric fluid, intestinal fluid, saliva, bile, tumor fluid, breast milk, urine, interstitial fluid, cerebral spinal fluid and stool. In some embodiments, the bodily fluid is blood. In some embodiments, the bodily fluid is plasma. In some embodiments, the bodily fluid is serum.

In some embodiments, the sample comprises a cancerous cell. In some embodiments, cancerous cell is a circulating tumor cell. In some embodiments, the cancerous cell is a melanoma cell. In some embodiments, the cancerous cell is a PD-L1 positive cell. In some embodiments, the cancerous cell is an HVEM positive cell.

In some embodiments, the sample comprises an immune cell. In some embodiments, the cell is a hematopoietic cell. In some embodiments, the immune cell is a T-cell. In some embodiments, the T-cell is a CD8 positive T-cell. In some embodiments, the T-cell is a cytotoxic CD8 positive T-cell. In some embodiments, the T-cell is a CD4 positive T-cell. In some embodiments, the T-cell is a CD4 positive helper T-cell. In some embodiments, the T-cell is selected from a CD8 positive and a CD4 positive T-cell. In some embodiments, the T-cell is a CD8 positive T-cell, a CD4 positive T-cell or both. In some embodiments, the immune cell is a tumor infiltrating lymphocyte (TIL). In some embodiments, the immune cell is not a peripheral blood immune cell. In some embodiments, the immune cell is a B-cell. In some embodiments, the immune cell is a natural killer (NK) cell. In some embodiments, the immune cell is a neutrophil. In some embodiments, the immune cell is a dendritic cell. In some embodiments, the immune cell is a macrophage. In some embodiments, the immune cell is a myeloid derived suppressor cell (MDSC). In some embodiments, the cell is selected from a T-cell, a B-cell, an NK cell, a neutrophil, a dendritic cell, an MDSC and a macrophage. In some embodiments, the cell is selected from a T-cell, a B-cell, an NK cell, a neutrophil, a dendritic cell, and a macrophage.

In some embodiments, the method comprises determining T cell levels in the sample. In some embodiments, T cell levels are T cell number. In some embodiments, determining T cell levels comprises counting T cells. In some embodiments, determining T cell levels comprises staining T cells. T cell markers and well known in the art and any may be used including, for example CD3. In some embodiments, determining T cell levels comprises measuring CD3 expression in the sample. In some embodiments, CD3 expression is CD3 levels. In some embodiments, T cell marker expression (e.g., CD3 expression) is proportional to T cell levels. In some embodiments, expression is protein expression. In some embodiments, expression is mRNA expression. In some embodiments, expression is surface expression. In some embodiments, the measuring comprises flow cytometry. In some embodiments, the flow cytometry is fluorescence-activated cell sorting (FACS). In some embodiments, the measuring comprises immunostaining. In some embodiments, the measuring comprises amplification. In some embodiments, measuring comprises sequencing. In some embodiments, the staining is staining of a T cell specific protein. In some embodiments the protein is a surface protein. In some embodiments, the amplification is amplification of a T cell specific mRNA. In some embodiments, the amplification is PCR. In some embodiments, the T cells are TILs. In some embodiments, the T cells are TILs and the determining is determining T cell levels in a tumor.

In some embodiments, determining expression comprises detecting expression. In some embodiments, determining expression comprises quantifying expression. In some embodiments, determining expression comprises detecting expression and quantifying the detected expression. Methods of measuring protein expression are well known in the art and include, for example, western blotting, immunohistochemistry, immunocytochemistry, immunofluorescent quantification, ELISA, FACS and protein arrays. Any such method may be employed. Methods of measuring mRNA/gene expression are also well known in the art and include, for example, northern blotting, RT-PCR, qPCR, microarray hybridization and sequencing (e.g., whole transcriptome sequencing). Any such method may be employed.

In some embodiments, the detecting comprises contacting the sample with an agent that specifically binds to HVEM. In some embodiments, the detecting comprises contacting the sample with an agent that binds HVEM mRNA. In some embodiments, the detecting comprises contacting the sample with an agent that binds HVEM DNA. In some embodiments, the agent specifically binds to HVEM. In some embodiments, the agent binds no other protein other than HVEM. In some embodiments, the agent binds an extracellular domain of HVEM. In some embodiments, the agent binds a cytoplasmic domain of HVEM. In some embodiments, the agent binds a transmembrane domain of HVEM. In some embodiments, detecting is detecting the extracellular domain of HVEM. In some embodiments, detecting is detecting the intracellular domain of HVEM. In some embodiments, detecting is detecting the transmembrane domain of HVEM. In some embodiments, detecting is detecting surface expression of HVEM. In some embodiments, detecting is detecting cytoplasmic expression of HVEM. In some embodiments, detecting is detecting surface and cytoplasmic expression of HVEM.

In some embodiments, human HVEM comprises or consist of the amino acid sequence MEPPGDWGPPPWRSTPKTDVLRLVLYLTFLGAPCYAPALPSCKEDEYPVGSECCP KCSPGYRVKEACGELTGTVCEPCPPGTYIAHLNGLSKCLQCQMCDPAMGLRASRN CSRTENAVCGCSPGHFCIVQDGDHCAACRAYATSSPGQRVQKGGTESQDTLCQNC PPGTFSPNGTLEECQHQTKCSWLVTKAGAGTSSSHWVWWFLSGSLVIVIVCSTVG LIICVKRRKPRGDVVKVIVSVQRKRQEAEGEATVIEALQAPPDVTTVAVEETIPSFT GRSPNH (SEQ ID NO: 17). In some embodiments, the signal peptide of HVEM comprises or consists of amino acids 1-38 of SEQ ID NO: 17. In some embodiments, the signal peptide of HVEM comprises or consists of amino acids 1-36 of SEQ ID NO: 17. In some embodiments, the HVEM extracellular domain comprises or consists of amino acids 39-202 of SEQ ID NO: 17. In some embodiments, the HVEM extracellular domain comprises or consists of amino acids 37-202 of SEQ ID NO: 17. In some embodiments, the HVEM transmembrane domain comprises or consists of amino acids 203-223 of SEQ ID NO: 17. In some embodiments, the HVEM cytoplasmic domain comprises or consists of amino acids 224-283 of SEQ ID NO: 17. In some embodiments, SEQ ID NO: 17 is a transmembrane form of HVEM. The generation of primers and/or probes that bind to these sequences is within the knowledge of a skilled artisan. Similarly, sequencing data which aligns with these sequences may be used to identify HVEM mRNA expression. It will be understood that protein sequences can comprise a certain level of variability within a population and thus any given human HVEM molecule may not be identical to SEQ ID NO: 17. In some embodiments, the HVEM comprises homology to SEQ ID NO: 17. In some embodiments, the HVEM extracellular domain comprises homology to amino acids 39-202 of SEQ ID NO: 17. In some embodiments, the HVEM extracellular domain comprises homology to amino acids 37-202 of SEQ ID NO: 17. In some embodiments, homology is at least 70, 75, 80, 85, 90, 92, 93, 95, 97, 98 or 99% homology. Each possibility represents a separate embodiment of the invention. In some embodiments, SEQ ID NO: 17 is isoform 1 of human HVEM. HVEM isoform 1 can be found in Uniprot entry Q92956-1.

In some embodiments, human HVEM comprises or consist of the amino acid sequence MEPPGDWGPPPWRSTPKTDVLRLVLYLTFLGAPCYAPALPSCKEDEYPVGSECCP KCSPGYRVKEACGELTGTVCEPCPPGTYIAHLNGLSKCLQCQMCDPAMGLRASRN CSRTENAVCGCSPGHFCIVQDGDHCAACRAYATSSPGQRVQKGGTESQDTLCQNC PPGTFSPNGTLEECQHQTK (SEQ ID NO: 18). In some embodiments, the signal peptide of HVEM comprises or consists of amino acids 1-38 of SEQ ID NO: 18. In some embodiments, the signal peptide of HVEM comprises or consists of amino acids 1-36 of SEQ ID NO: 18. In some embodiments, SEQ ID NO: 18 is a soluble form of HVEM. In some embodiments, SEQ ID NO: 18 is a form of HVEM lacking a transmembrane and cytoplasmic domain. In some embodiments, the HVEM extracellular domain comprises or consists of SEQ ID NO: 18. The generation of primers and/or probes that bind to these sequences is within the knowledge of a skilled artisan. Similarly, sequencing data which aligns with these sequences may be used to identify HVEM mRNA expression. It will be understood that protein sequences can comprise a certain level of variability within a population and thus any given human HVEM molecule may not be identical to SEQ ID NO: 18. In some embodiments, the HVEM comprises homology to SEQ ID NO: 18. In some embodiments, the HVEM extracellular domain comprises homology to amino acids 37-202 of SEQ ID NO: 18. In some embodiments, homology is at least 70, 75, 80, 85, 90, 92, 93, 95, 97, 98 or 99% homology. Each possibility represents a separate embodiment of the invention. In some embodiments, SEQ ID NO: 18 is isoform 2 of human HVEM.

An alternative isoform of HVEM is also known in which the extracellular domain is truncated. This isoform can be found in Uniprot entry Q92956-2. In some embodiments, and extracellular domain truncated human HVEM comprises or consist of the amino acid sequence MVSRPPRTPLSPSSWTPAMGLRASRNCSRTENAVCGCSPGHFCIVQDGDHCAACR AYATSSPGQRVQKGGTESQDTLCQNCPPGTFSPNGTLEECQHQTKCSWLVTKAGA GTSSSHWVWWFLSGSLVIVIVCSTVGLIICVKRRKPRGDVVKVIVSVQRKRQEAEG EATVIEALQAPPDVTTVAVEETIPSFTGRSPNH (SEQ ID NO: 19). In some embodiments, the signal peptide of HVEM comprises or consists of amino acids 1-16 of SEQ ID NO: 19. In some embodiments, the HVEM extracellular domain comprises or consists of amino acids 17-118 of SEQ ID NO: 19. In some embodiments, the HVEM transmembrane domain comprises or consists of amino acids 119-139 of SEQ ID NO: 19. In some embodiments, the HVEM cytoplasmic domain comprises or consists of amino acids 140-199 of SEQ ID NO: 19.In some embodiments, SEQ ID NO: 19 is a transmembrane form of HVEM. In some embodiments, SEQ ID NO: 19 is a form of HVEM comprising a truncated extracellular domain. The generation of primers and/or probes that bind to these sequences is within the knowledge of a skilled artisan. Similarly, sequencing data which aligns with these sequences may be used to identify HVEM mRNA expression. It will be understood that protein sequences can comprise a certain level of variability within a population and thus any given human HVEM molecule may not be identical to SEQ ID NO: 19. In some embodiments, the HVEM comprises homology to SEQ ID NO: 19. In some embodiments, the HVEM extracellular domain comprises homology to amino acids 17-118 of SEQ ID NO: 19. In some embodiments, homology is at least 70, 75, 80, 85, 90, 92, 93, 95, 97, 98 or 99% homology. Each possibility represents a separate embodiment of the invention.

In some embodiments, the agent is an antibody or antigen binding fragment thereof. In some embodiments, the antibody or fragment thereof is a fab fragment. In some embodiments, the antibody or fragment thereof is a single chain antibody (scFv). In some embodiments, the antibody or fragment thereof is a single domain antibody. In some embodiments, the antibody is a human antibody. In some embodiments, the antibody is a rodent antibody. In some embodiments, the antibody is a goat antibody. In some embodiments, the antibody is a rabbit antibody. In some embodiments, the antibody is a humanized antibody. In some embodiments, the antibody is a monoclonal antibody. In some embodiments, the antibody is a polyclonal antibody.

As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. An antibody may be oligoclonal, polyclonal, monoclonal, chimeric, camelised, CDR-grafted, multi- specific, bi-specific, catalytic, humanized, fully human, anti- idiotypic and antibodies that can be labeled in soluble or bound form as well as fragments, including epitope-binding fragments, variants or derivatives thereof, either alone or in combination with other amino acid sequences. An antibody may be from any species. The term antibody also includes binding fragments, including, but not limited to Fv, Fab, Fab′, F(ab′)2 single stranded antibody (svFC), dimeric variable region (Diabody) and disulphide-linked variable region (dsFv). In particular, antibodies include immunoglobulin molecules and immunologically active fragments of immunoglobulin molecules, i.e., molecules that contain an antigen binding site. Antibody fragments may or may not be fused to another immunoglobulin domain including but not limited to, an Fc region or fragment thereof. The skilled artisan will further appreciate that other fusion products may be generated including but not limited to, scFv- Fc fusions, variable region (e.g., VL and VH)~ Fc fusions and scFv-scFv-Fc fusions.

Immunoglobulin molecules can be of any type (e.g., IgG, IgE, IgM, IgD, IgA and IgY), class (e.g., IgG1, IgG2, IgG3, IgG4, IgAl and IgA2) or subclass. In some embodiments, the antibody comprises IgG2 or IgG4. In some embodiments, the antibody comprises IgG2. In some embodiments, the antibody comprises IgG4. In some embodiments, the antibody comprises IgG1. In some embodiments, the antibody comprises IgG3. In some embodiments, the antibody comprises a modified IgG1 or IgG3 with reduced toxicity.

The basic unit of the naturally occurring antibody structure is a heterotetrameric glycoprotein complex of about 150,000 Daltons, composed of two identical light (L) chains and two identical heavy (H) chains, linked together by both noncovalent associations and by disulfide bonds. Each heavy and light chain also has regularly spaced intra-chain disulfide bridges. Five human antibody classes (IgG, IgA, IgM, IgD and IgE) exist, and within these classes, various subclasses, are recognized based on structural differences, such as the number of immunoglobulin units in a single antibody molecule, the disulfide bridge structure of the individual units, and differences in chain length and sequence. The class and subclass of an antibody is its isotype.

The amino terminal regions of the heavy and light chains are more diverse in sequence than the carboxy terminal regions, and hence are termed the variable domains. This part of the antibody structure confers the antigen-binding specificity of the antibody. A heavy variable (VH) domain and a light variable (VL) domain together form a single antigen-binding site, thus, the basic immunoglobulin unit has two antigen-binding sites. Particular amino acid residues are believed to form an interface between the light and heavy chain variable domains (Chothia et al., J. Mol. Biol. 186, 651-63 (1985); Novotny and Haber, (1985) Proc. Natl. Acad. Sci. USA 82 4592-4596).

The carboxy terminal portion of the heavy and light chains form the constant domains i.e., CH1, CH2, CH3, CL. While there is much less diversity in these domains, there are differences from one animal species to another, and further, within the same individual there are several different isotypes of antibody, each having a different function.

The term “framework region” or “FR” refers to the amino acid residues in the variable domain of an antibody, which are other than the hypervariable region amino acid residues as herein defined. The term “hypervariable region” as used herein refers to the amino acid residues in the variable domain of an antibody, which are responsible for antigen binding. The hypervariable region comprises amino acid residues from a “complementarity determining region” or “CDR”. The CDRs are primarily responsible for binding to an epitope of an antigen. The extent of FRs and CDRs has been precisely defined (see, Kabat et al.).

Immunoglobulin variable domains can also be analyzed using the IMGT information system (www://imgt. cines.fr/) (IMGT®/V-Quest) to identify variable region segments, including CDRs. See, e.g., Brochet, X. et al, Nucl. Acids Res. J6:W503-508 (2008).

As used herein, the term “humanized antibody” refers to an antibody from a non-human species whose protein sequences have been modified to increase similarity to human antibodies. A humanized antibody may be produced by production of recombinant DNA coding for the CDRs of the non-human antibody surrounded by sequences that resemble a human antibody. In some embodiments, the humanized antibody is a chimeric antibody. In some embodiments, humanizing comprises insertion of the CDRs of the invention into a human antibody scaffold or backbone. Humanized antibodies are well known in the art and any method of producing them that retains the CDRs of the invention may be employed.

The term “monoclonal antibody” or “mAb” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. The term “polyclonal antibody” as used herein refers to a mix of heterogenous antibodies. In contrast to polyclonal antibody preparations that typically include different antibodies directed against different determinants (epitopes), a monoclonal antibody is directed against a single determinant on the antigen. In addition to their specificity, the monoclonal antibodies are advantageous in that they are uncontaminated by other immunoglobulins. The modifier “monoclonal” indicates the character of the antibody as being obtained from a substantially homogeneous population of antibodies and is not to be construed as produced by any specific preparation method. Monoclonal antibodies to be used in accordance with the methods provided herein, may be made by the hybridoma method first described by Kohler et al, Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al, Nature 352:624-628 (1991) and Marks et al, J. Mol. Biol. 222:581-597 (1991), for example.

The mAb of the present invention may be of any immunoglobulin class including IgG, IgM, IgD, IgE or IgA. A hybridoma producing a mAb may be cultivated in vitro or in vivo. High titers of mAbs can be obtained in vivo production where cells from the individual hybridomas are injected intraperitoneally into pristine-primed Balb/c mice to produce ascites fluid containing high concentrations of the desired mAbs. mAbs of isotype IgM or IgG may be purified from such ascites fluids, or from culture supernatants, using column chromatography methods well known to those of skill in the art.

“Antibody fragments” comprise a portion of an intact antibody, preferably comprising the antigen binding region thereof. Examples of antibody fragments include Fab, Fab′, F(ab′)2, and Fv fragments; diabodies; tandem diabodies (taDb), linear antibodies (e.g., U.S. Pat. No. 5,641,870, Example 2; Zapata et al, Protein Eng. 8(10): 1057-1062 (1995)); one-armed antibodies, single variable domain antibodies, minibodies, single-chain antibody molecules; multispecific antibodies formed from antibody fragments (e.g., including but not limited to, Db- Fc, taDb-Fc, taDb-CH3, (scFV)4-Fc, di-scFv, bi-scFv, or tandem (di,tri)-scFv); and Bi-specific T-cell engagers (BiTEs).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-binding sites and is still capable of cross-linking antigen.

“Fv” is the minimum antibody fragment that contains a complete antigen-recognition and antigen-binding site. This region consists of a dimer of one heavy chain and one light chain variable domain in tight, non-covalent association. It is in this configuration that the three surfaces of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)2 antibody fragments originally were produced as pairs of Fab′ fragments that have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy chain constant domains that correspond to the different classes of antibodies are called a, delta, e, gamma, and micro, respectively. The subunit structures and three-dimensional configurations of different classes of immunoglobulins are well known.

“Single-chain Fv” or “scFv” antibody fragments comprise the VH and VL domains of antibody, wherein these domains are present in a single polypeptide chain. In some embodiments, the Fv polypeptide further comprises a polypeptide linker between the VH and VL domains that enables the scFv to form the desired structure for antigen binding. For a review of scFv see Pluckthun in The Pharmacology of Monoclonal Antibodies, vol. 113, Rosenburg and Moore eds., Springer- Verlag, New York, pp. 269-315 (1994).

The term “diabodies” refers to small antibody fragments with two antigen-binding sites, which fragments comprise a heavy chain variable domain (VH) connected to a light chain variable domain (VL) in the same polypeptide chain (VH - VL). By using a linker that is too short to allow pairing between the two domains on the same chain, the domains are forced to pair with the complementary domains of another chain and create two antigen-binding sites. Diabodies production is known in the art and is described in Natl. Acad. Sci. USA, 90:6444-6448 (1993).

The monoclonal antibodies of the invention may be prepared using methods well known in the art. Examples include various techniques, such as those in Kohler, G. and Milstein, C, Nature 256: 495-497 (1975); Kozbor et al, Immunology Today 4: 72 (1983); Cole et al, pg. 77-96 in MONOCLONAL ANTIBODIES AND CANCER THERAPY, Alan R. Liss, Inc. (1985).

Besides the conventional method of raising antibodies in vivo, antibodies can be generated in vitro using phage display technology. Such a production of recombinant antibodies is much faster compared to conventional antibody production and they can be generated against an enormous number of antigens. Furthermore, when using the conventional method, many antigens prove to be non-immunogenic or extremely toxic, and therefore cannot be used to generate antibodies in animals. Moreover, affinity maturation (i.e., increasing the affinity and specificity) of recombinant antibodies is very simple and relatively fast. Finally, large numbers of different antibodies against a specific antigen can be generated in one selection procedure. To generate recombinant monoclonal antibodies, one can use various methods all based on display libraries to generate a large pool of antibodies with different antigen recognition sites. Such a library can be made in several ways: One can generate a synthetic repertoire by cloning synthetic CDR3 regions in a pool of heavy chain germline genes and thus generating a large antibody repertoire, from which recombinant antibody fragments with various specificities can be selected. One can use the lymphocyte pool of humans as starting material for the construction of an antibody library. It is possible to construct naive repertoires of human IgM antibodies and thus create a human library of large diversity. This method has been widely used successfully to select a large number of antibodies against different antigens. Protocols for bacteriophage library construction and selection of recombinant antibodies are provided in the well-known reference text Current Protocols in Immunology, Colligan et al (Eds.), John Wiley & Sons, Inc. (1992-2000), Chapter 17, Section 17.1.

Non-human antibodies may be humanized by any methods known in the art. In one method, the non-human complementarity determining regions (CDRs) are inserted into a human antibody or consensus antibody framework sequence. Further changes can then be introduced into the antibody framework to modulate affinity or immunogenicity.

In some embodiments, antibodies and portions thereof include but are not limited to: antibodies, fragments of antibodies, Fab and F(ab′)2, single-domain antigen-binding recombinant fragments and natural nanobodies. In some embodiments, the antigen binding fragment is selected from the group consisting of a Fv, Fab, F(ab′)₂, scFv, scFv₂ or a scFv₄ fragment.

In some embodiments, the agent is an antibody or antigen binding fragment thereof comprises three heavy chain CDRs (CDR-H) and three light chain CDRs (CDR-L), wherein: CDR-H1 comprises the amino acid sequence set forth in SEQ ID NO: 1 (SYAMS), CDR-H2 comprises the amino acid sequence as set forth in SEQ ID NO: 2 (AISGSGGSTYYADSVKG), CDR-H3 comprises the amino acid sequence as set forth in SEQ ID NO: 3 (APGDYTAYFDY), CDR-L1 comprises the amino acid sequence as set forth in SEQ ID NO: 4 (RASQSVSSYLA), CDR-L2 comprises the amino acid sequence as set forth in SEQ ID NO: 5 (GASSRAT), and CDR-L3 comprises the amino acid sequence as set forth in SEQ ID NO: 6 (QQYGSSPPYT). In some embodiments, the CDRs are according to the KABAT numbering system.

In some embodiments, the antibody or antigen binding fragment thereof comprises three heavy chain CDRs (CDR-H) and three light chain CDRs (CDR-L), wherein: CDR-H1 comprises the amino acid sequence set forth in SEQ ID NO: 11 (GFTFSSYA), CDR-H2 comprises the amino acid sequence as set forth in SEQ ID NO: 12 (ISGSGGST), CDR-H3 comprises the amino acid sequence as set forth in SEQ ID NO: 13 (AKAPGDYTAYFDY), CDR-L1 comprises the amino acid sequence as set forth in SEQ ID NO: 14 (QSVSSY), CDR-L2 comprises the amino acid sequence as set forth in SEQ ID NO: 15 (GAS), and CDR-L3 comprises the amino acid sequence as set forth in SEQ ID NO: 16 (QQYGSSPPYT). In some embodiments, the CDRs are according to the IMGT numbering system.

In some embodiments, the agent is an antibody or antigen binding fragment thereof comprises three heavy chain CDRs (CDR-H) and three light chain CDRs (CDR-L), wherein: CDR-H1 comprises the amino acid sequence set forth in SEQ ID NO: 11 (GFTFSSYA), CDR-H2 comprises the amino acid sequence as set forth in SEQ ID NO: 12 (ISGSGGST), CDR-H3 comprises the amino acid sequence as set forth in SEQ ID NO: 13 (AKAPGDYTAYFDY), CDR-L1 comprises the amino acid sequence as set forth in SEQ ID NO: 14 (QSVSSY), CDR-L2 comprises the amino acid sequence as set forth in SEQ ID NO: 15 (GAS), and CDR-L3 comprises the amino acid sequence as set forth in SEQ ID NO: 16 (QQYGSSPPYT). In some embodiments, the CDRs are according to the IMGT numbering system.

In some embodiments, the antibody comprises a heavy chain comprising the sequence QVQLVQSGGGLVQPGGSLRLSCAASGFTFSSYAMSWVRQAPGKGLEWVSAISGS GGSTYYADSVKGRFTISRDNSKNTLYLQMNSLRAEDTAVYYCAKAPGDYTAYFD YWGQGTLVTVSS (SEQ ID NO: 7). In some embodiments, the heavy chain consists of the sequence of SEQ ID NO: 7. In some embodiments, the antibody comprises a heavy chain comprising the sequence MGWSCIILFLVATATGVHSQVQLVQSGGGLVQPGGSLRLSCAASGFTFSSYAMSW VRQAPGKGLEWVSAISGSGGSTYYADSVKGRFTISRDNSKNTLYLQMNSLRAEDT AVYYCAKAPGDYTAYFDYWGQGTLVTVSSASTKGPSVFPLAPCSRSTSESTAALG CLVKDYFPEPVTVSWNSGALTSGVHTFPAVLQSSGLYSLSSVVTVPSSSLGTKTYT CNVDHKPSNTKVDKRVESKYGPPCPPCPAPEFLGGPSVFLFPPKPKDTLMISRTPEV TCVVVDVSQEDPEVQFNWYVDGVEVHNAKTKPREEQFNSTYRVVSVLTVLHQD WLNGKEYKCKVSNKGLPSSIEKTISKAKGQPREPQVYTLPPSQEEMTKNQVSLTCL VKGFYPSDIAVEWESNGQPENNYKTTPPVLDSDGSFFLYSRLTVDKSRWQEGNVF SCSVMHEALHNHYTQKSLSLSLGK (SEQ ID NO: 9). In some embodiments, the heavy chain consists of the sequence of SEQ ID NO: 9.

In some embodiments, the antibody comprises a light chain comprising the sequence ELVLTQSPATLSLSPGERATLSCRASQSVSSYLAWYQQKPGQAPRLLIYGASSRAT GIPDRFSGSGSGTDFTLTISRLEPEDFAVYYCQQYGSSPPYTFGQGTKVEIK (SEQ ID NO: 8). In some embodiments, the light chain consists of the sequence of SEQ ID NO: 8. In some embodiments, the antibody comprises a light chain comprising the sequence MGWSCIILFLVATATGVHSELVLTQSPATLSLSPGERATLSCRASQSVSSYLAWYQ QKPGQAPRLLIYGASSRATGIPDRFSGSGSGTDFTLTISRLEPEDFAVYYCQQYGSS PPYTFGQGTKVEIKRTVAAPSVFIFPPSDEQLKSGTASVVCLLNNFYPREAKVQWK VDNALQSGNSQESVTEQDSKDSTYSLSSTLTLSKADYEKHKVYACEVTHQGLSSP VTKSFNRGEC (SEQ ID NO: 10). In some embodiments, the light chain consists of the sequence of SEQ ID NO: 10.

In some embodiments, the method further comprises administering the PD-1/PD-L1 based immunotherapy. In some embodiments, administering is administering a therapeutically effective amount of the immunotherapy. The term “therapeutically effective amount” refers to an amount of a drug effective to treat a disease or disorder in a mammal. In some embodiments, a therapeutically effective amount is an amount effective, at dosages and for periods of time necessary, to achieve the desired therapeutic or prophylactic result. The exact dosage form and regimen would be determined by the physician according to the patient’s condition.

In some embodiments, the detecting comprises contacting the sample with an agent that binds to HVEM. In some embodiments, the detecting comprises contacting the sample with an agent that binds HVEM mRNA. In some embodiments, the detecting comprises contacting the sample with an agent that binds HVEM DNA. In some embodiments, the contacting is under conditions suitable for binding of the agent or antibody or antigen binding fragment thereof to bind to HVEM. In some embodiments, the conditions are suitable for specific binding to HVEM. In some embodiments, binding is hybridizing. Conditions for antibody/agent binding will depend on the sample. A skilled artisan will appreciate the conditions needed for binding to a tissue sample (dependent on the method/type of fixation) are different than those of binding in a liquid solution, such as a whole cell lysate. Such binding conditions are well known in the art and a skilled artisan can select the proper conditions for a given sample.

In some embodiments, the detection is immunohistochemical detection. In some embodiments, the detection is immunocytochemical detection. In some embodiments, the detection is immunofluorescent detection. In some embodiments, the detection is FACS detection. In some embodiments, the detection further comprises contacting the sample with a secondary agent that recognized the agent or antibody. In some embodiments, the detection comprises detecting the secondary agent. In some embodiments, the secondary agent is an antibody. In some embodiments, the secondary agent comprises a detectable moiety. In some embodiments, agent comprises a detectable moiety. In some embodiments, the detecting comprises detecting the detectable moiety. In some embodiments, the detection comprises microscopy. In some embodiments, the detection comprises flow cytometry. In some embodiments, the detection comprises FACS. In some embodiments, the detection comprises ELISA.

In some embodiments, the subject suffers from a disease or condition. In some embodiments, the subject is suspected to suffer from a disease or condition. In some embodiments, the subject is at risk of developing a disease or condition. In some embodiments, the subject suffers from a disease or condition which may comprise increased HVEM expression. In some embodiments, the subject suffers from a disease or condition which may be characterized by increased HVEM expression. In some embodiments, the subject suffers from cancer. In some embodiments, the cancer is a cancer that did not respond to first line therapy. In some embodiments, the cancer is a cancer relapse. In some embodiments, the cancer is a cancer that did not respond to PD-1/PD-L1 based immunotherapy.

In some embodiments, positive expression of HVEM comprises HVEM expression. In some embodiments, positive expression of HVEM comprises elevated HVEM levels. In some embodiments, positive expression of HVEM comprises increased HVEM levels. In some embodiments, positive expression of HVEM comprises overexpression of HVEM. In some embodiments, positive expression of HVEM is as compared to healthy sample. In some embodiments, the healthy sample is from healthy tissue and/or cells adjacent to the disease tissue and/or cells. In some embodiments, a healthy sample comprises non-infected cells. In some embodiments, a healthy sample is for a healthy donor. In some embodiments, positive expression of HVEM is as compared to a predetermined threshold. In some embodiments, elevated or overexpression is by at least 5, 10, 15, 20, 25, 30, 35, 40, 45, 50, 55, 60, 70, 75, 80, 85, 90, 95, 100, 125, 150, 175, 200, 250, 300, 350, 400, 450, or 500%. Each possibility represents a separate embodiment of the invention.

In some embodiments, HVEM expression is HVEM mRNA expression and the detecting comprises detecting HVEM mRNA. In some embodiments, the detecting is detecting specifically HVEM mRNA. Methods of mRNA detecting are well known in the art and may include for example, northern blotting, PCR, microarrays and sequencing. Any such method may be employed. In some embodiments, detecting comprises PCR. In some embodiments, the PCR is quantitative PCR (qPCR). In some embodiments, the PCR is real-time PCR. In some embodiments, the detecting comprises sequencing. In some embodiments, the sequencing is deep sequencing. In some embodiments, the sequencing is next generation sequencing. In some embodiments, the sequencing is whole transcriptome sequencing. Examples of HVEM primers or probes are well known in the art and any such sequence or molecule may be employed. Information of the human HVEM gene can be found in Entrez gene entry 8764. The sequence of human HVEM isoform 1 mRNA is provided in RefSeq NM_003820. The sequence of human HVEM isoform 1 protein is provided in RefSeq NP_ 003811 and SEQ ID NO: 17. An alternative HVEM isoform (isoform 2) is also known. This isoform lacks a transmembrane domain and thus is a soluble form of HVEM. HVEM isoform 2′s mRNA sequence is provided in RefSeq NM_001297605 and its amino acid sequence is provided in RefSeq NP_001284534 and SEQ ID NO: 18.

In some embodiments, the sample is a tissue sample. In some embodiments, the sample is a paraffin embedded sample. In some embodiments, the sample is a perfused sample. In some embodiments, the sample is from the subject. In some embodiments, the sample is a healthy sample. In some embodiments, the sample is a diseased sample. In some embodiments, the sample is a diagnostic sample. In some embodiments, the sample is a biological fluid. In some embodiments, the biological fluid is selected from blood, serum, plasma, urine, feces, bile, semen, tumor fluid, and cerebral spinal fluid. In some embodiments, the sample is a blood sample. In some embodiments, the sample is a cell sample. In some embodiments, the sample comprises cells. In some embodiments, the sample comprises disease cells. In some embodiments, the sample is a tumor sample. In some embodiments, the sample is a biopsy.

In some embodiments, the method further comprises treating a suitable subject. In some embodiments, the treating is with the PD-1 based immunotherapy. In some embodiments, the treating is with the PD-L1 based immunotherapy. In some embodiments, the treating is with the PD-1/PD-L1 based immunotherapy. In some embodiments, the treating comprises administering the immunotherapy. In some embodiments, the treating is with a HVEM based immunotherapy. In some embodiments, the treating is with a combined PD-1/PD-L1 based immunotherapy and an HVEM based immunotherapy.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition or method herein needs only to reduce the severity of a disease, disorder, or condition, reduce the severity of symptoms associated therewith, or provide improvement to a patient or subject’s quality of life.

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of a composition of the present subject matter to a patient in need thereof. Other suitable routes of administration can include parenteral, subcutaneous, oral, intramuscular, topical or intraperitoneal. In some embodiments, the administration is systemic administration. In some embodiments, the administration is local administration.

The dosage administered will be dependent upon the age, health, and weight of the recipient, kind of concurrent treatment, if any, frequency of treatment, and the nature of the effect desired.

By another aspect, there is provided an agent that specifically detects HVEM for use in a method of the invention.

By another aspect, there is provided a kit comprising an agent that specifically detects HVEM for use in a method of the invention.

By another aspect, there is provided an agent that specifically detects T cells for use in a method of the invention.

By another aspect, there is provided a kit comprising an agent that specifically detects T cells for use in a method of the invention.

In some embodiments, the kit further comprises a secondary detection molecule. In some embodiments, the detection molecule is for detection of the agent. In some embodiments, the secondary detection molecule is an antibody that binds to the agent. In some embodiments, the detection molecule comprises a detectable moiety. Examples of detectable moieties include, but are not limited to a fluorescent moiety, a tag, a radiolabel, a dye and a chemiluminescent moiety. In some embodiments, the detectable moiety is a fluorescent moiety. Examples of fluorescent moieties include, but are not limited to GFP, RFP, YFP, APC, CY5, CY7, and Pacific Blue. Secondary detection molecules are well known in the art and any such molecule that will detect an agent of the invention may be used.

In some embodiments, agent specifically binds to HVEM protein. In some embodiments, the agent specifically binds to HVEM mRNA. In some embodiments, the agent specifically binds to an HVEM cDNA. In some embodiments, the agent is a primer. In some embodiments, the agent is a nucleic acid probe. In some embodiments, the agent is an antibody or antigen binding fragment thereof.

As used herein, the term “about” when combined with a value refers to plus and minus 10% of the reference value. For example, a length of about 1000 nanometers (nm) refers to a length of 1000 nm+- 100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all subcombinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells - A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization - A Laboratory Course Manual” CSHL Press (1996); all of which are incorporated by reference. Other general references are provided throughout this document.

### Example 1: Immunohistochemistry on Tumor Microarrays

HVEM and PD-L1 are both immune checkpoint proteins expressed in the plasma membrane of many cancers. It has been suggested (Malissen et al., 2019, “HVEM has a broader expression than PD-L1 and constitutes a negative prognostic marker and potential treatment target for melanoma”, OncoImmunology, 8:12, e1665976) that high expression of HVEM is a marker for poor prognosis. Others have suggested that circulating HVEM levels do not correlate with immunotherapy response (Zhang et al., 2020, “Anti-PD-1 therapy response predicted by the combination of exosomal PD-L1 and CD28” Front. Oncol., 10: 760). In order to determine the relative expression of these two markers, Tumor MicroArrays (TMAs) of melanoma cells were selected containing paraffin embedded tumor samples from both responders and non-responders to PD-1 blockade (Nivolumab or Pembrolizumab). The TMAs contained 20 patients characterized as responders and 16 patients characterized as non-responders. The TMAs contained 1-3 cores from each of these patients.

5 µm sections were prepared from each block, deparaffinized, and antigen retrieval was performed in a basic solution (pH 9). Triple staining was performed using a mouse monoclonal anti-CD3 antibody (Proteintech, #60181-1-Ig; 5 µg/ml), a rabbit monoclonal anti-PD-L1 antibody (Cell Signaling, #13684, 10 µg/ml) and a goat polyclonal anti-HVEM IgG antibody (R&D Systems #AF356, 10 µg/ml) as primary antibodies. Anti-mouse Cy2, anti-rabbit Cy3 and anti-goat Cy5 secondary antibodies were used (Jackson, USA; #715-545-151/152/147 respectively, 3.125 µg/ml) and nuclei were stained with DAPI. In addition to the TMAs, T cells (primary TILs) and PD-L1 positive melanoma cells were also stained. Imaging was performed with a Leica TCS SP5 confocal laser-scanning microscope (X63 magnification) and image analysis was performed using the FIJI (Image J2) software.

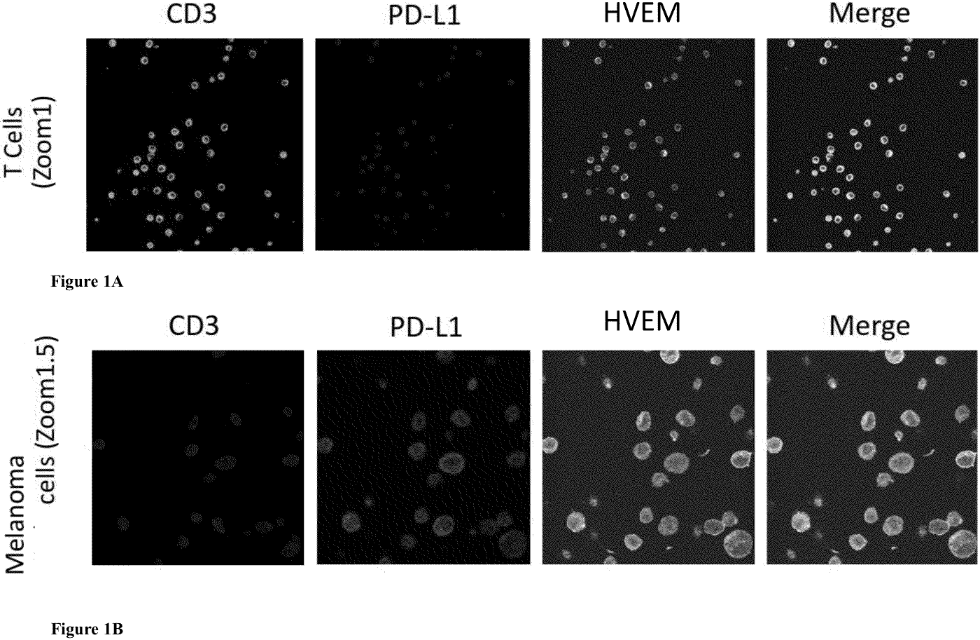

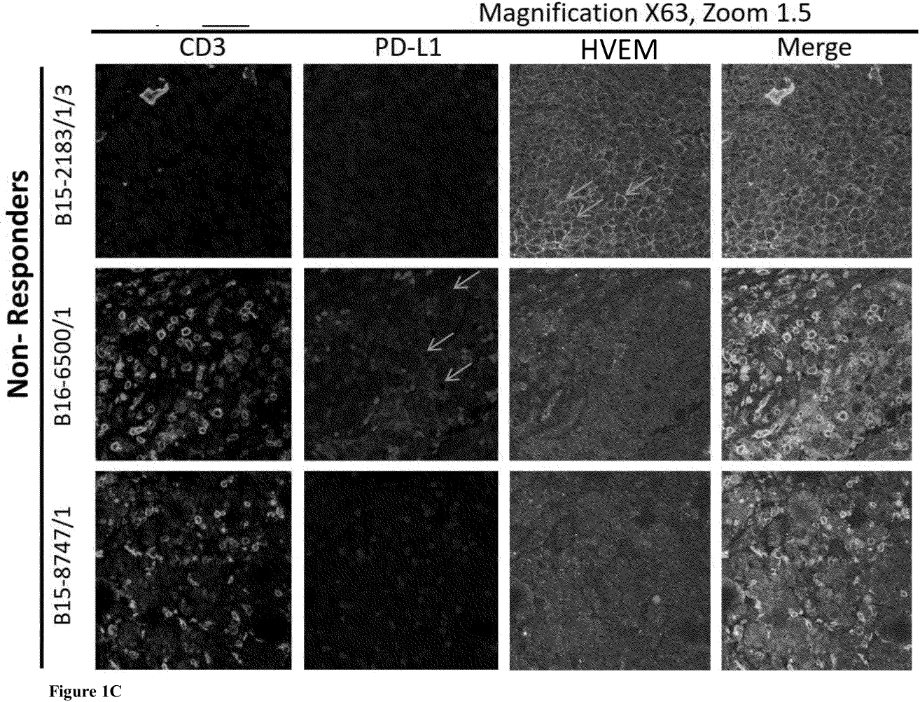

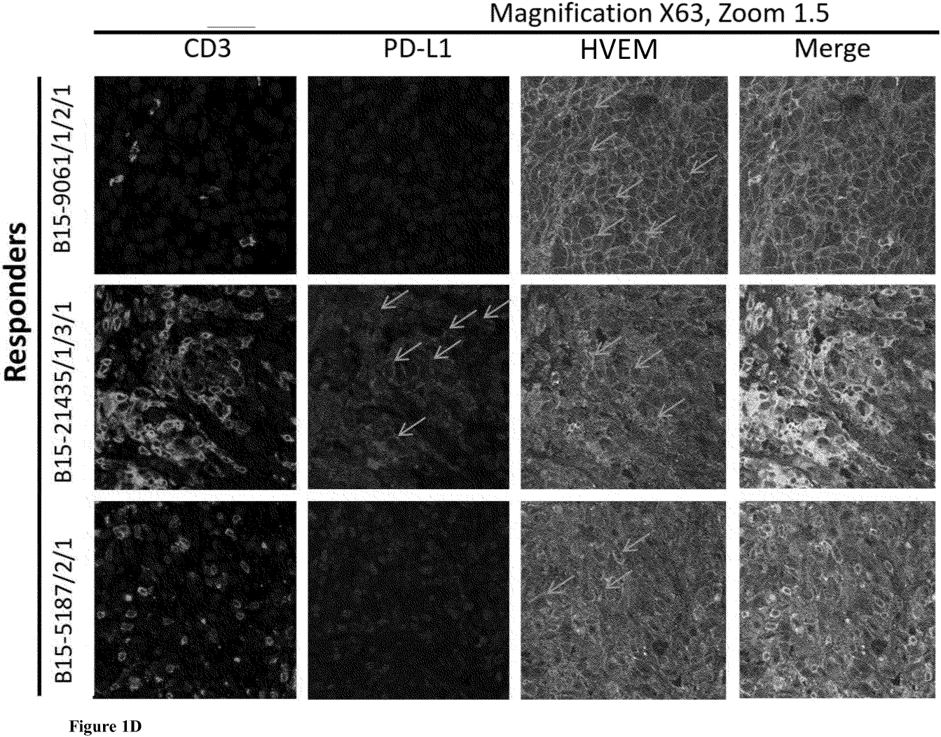

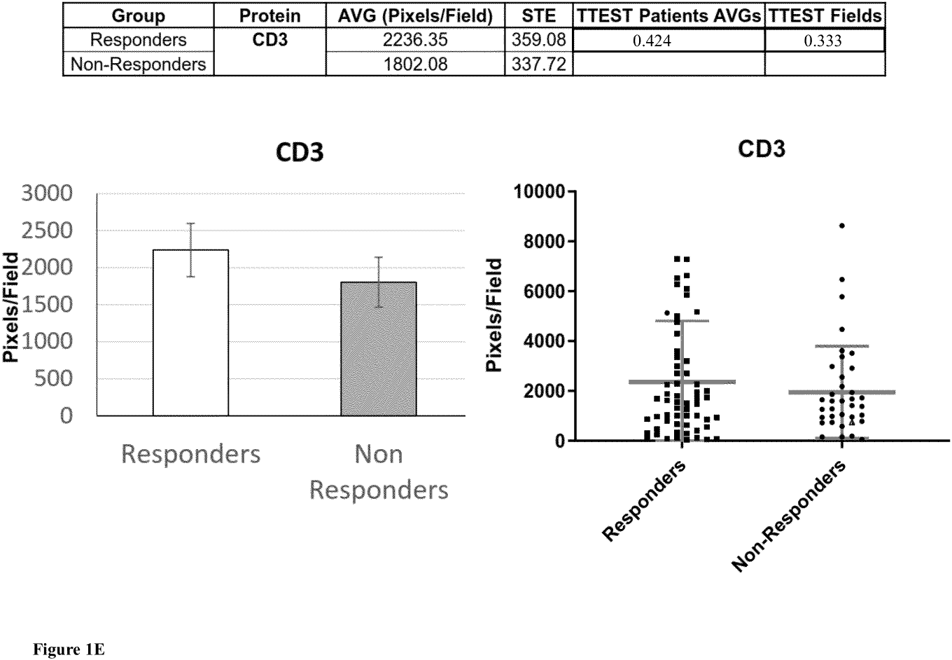

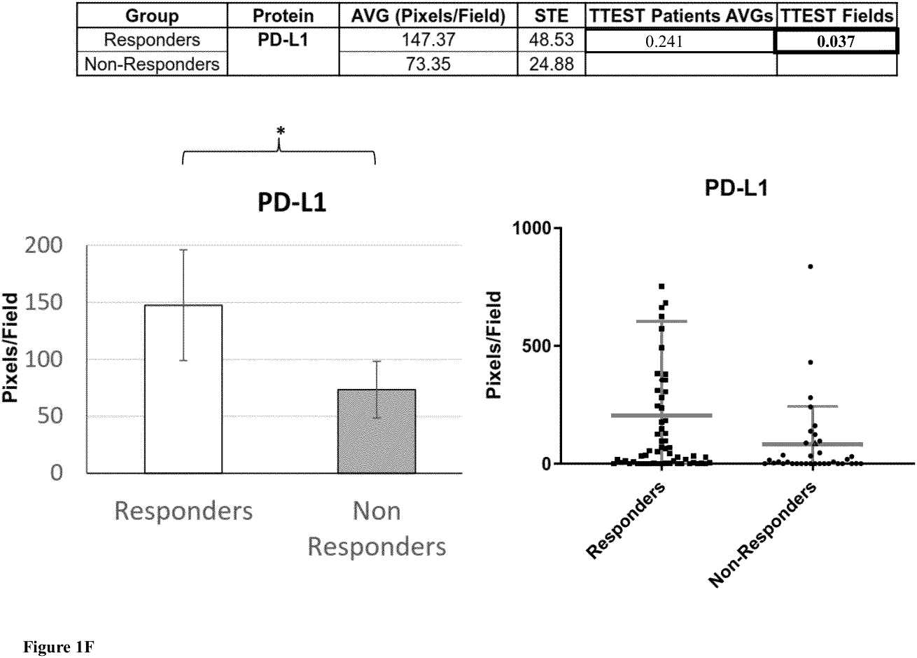

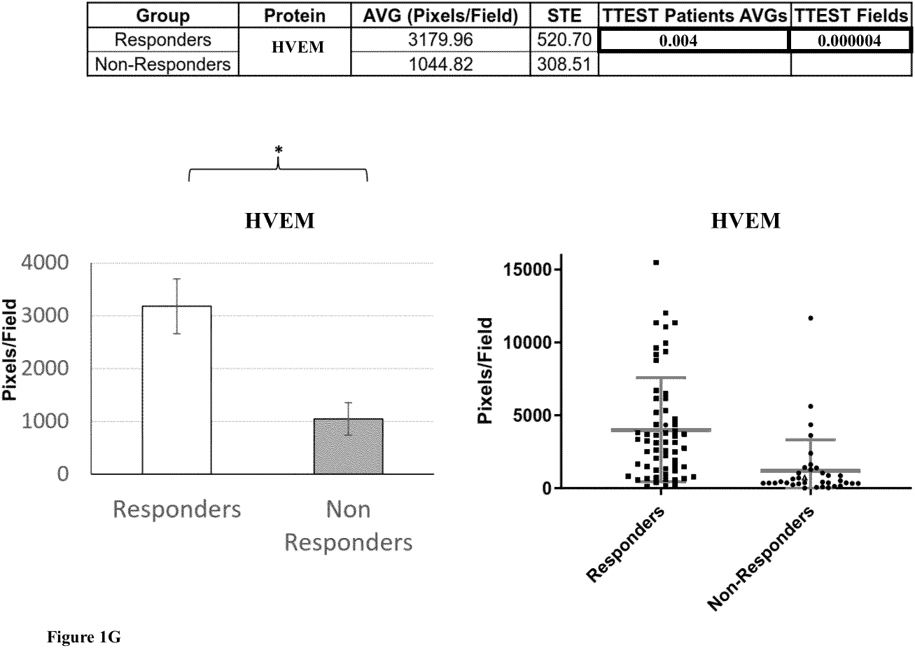

As expected, the control T cells were positive for CD3, to a lesser extent HVEM and were negative for PD-L1 (FIG. 1A, cell surface expression). The melanoma cells were negative for CD3, and strongly positive for PD-L1 and HVEM (FIG. 1B, mostly cell surface expression for both, with some HVEM cytoplasmic expression). Staining of non-responders found a high degree of heterogeneity among the samples, with CD3, PD-L1 and HVEM all ranging from negative in some samples to highly expressed in others (FIG. 1C). The same heterogeneity was observed in the responder samples (FIG. 1D). However, quantification of the expression of each protein over the entire population of responders and non-responders yielded both expected and unexpected results. CD3 expression was comparable between the two populations and the small difference observed was not statistically significant (FIG. 1E). PD-L1 expression was elevated in the responder population and this increase was significant when all the fields from all the patients in the group were analyzed (FIG. 1F). This PD-L1 result has been reported before and is easy to understand as subjects with the highest expression of the target of the therapeutic molecule are the most likely to responds to that therapeutic molecule. Finally, an examination of HVEM expression showed a most unexpected result; although HVEM has been asserted in the literature as a marker for poor prognosis, higher levels of HVEM were observed in the sample in the responders group as compared to the non-responders (FIG. 1G). This result was statistically significant when all the images were assessed and even when the average for each subject was used in the calculation. This result indicates that HVEM expression rather than being a negative prognostic marker is actually a positive prognostic marker for response to PD-1/PD-L1 based immunotherapies and can be used diagnostically to determine a patient population that is more likely to respond to these immunotherapies.

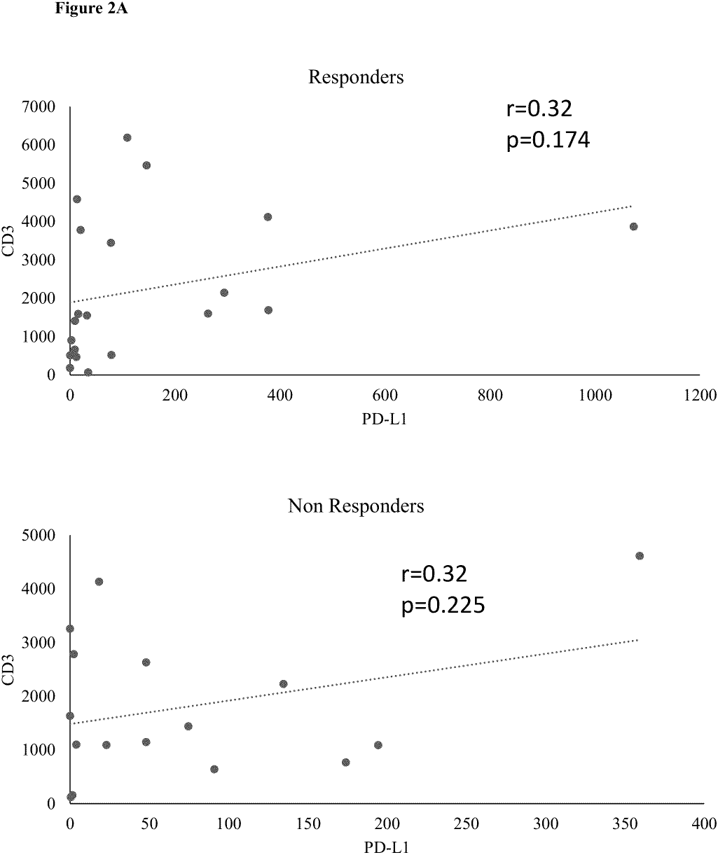

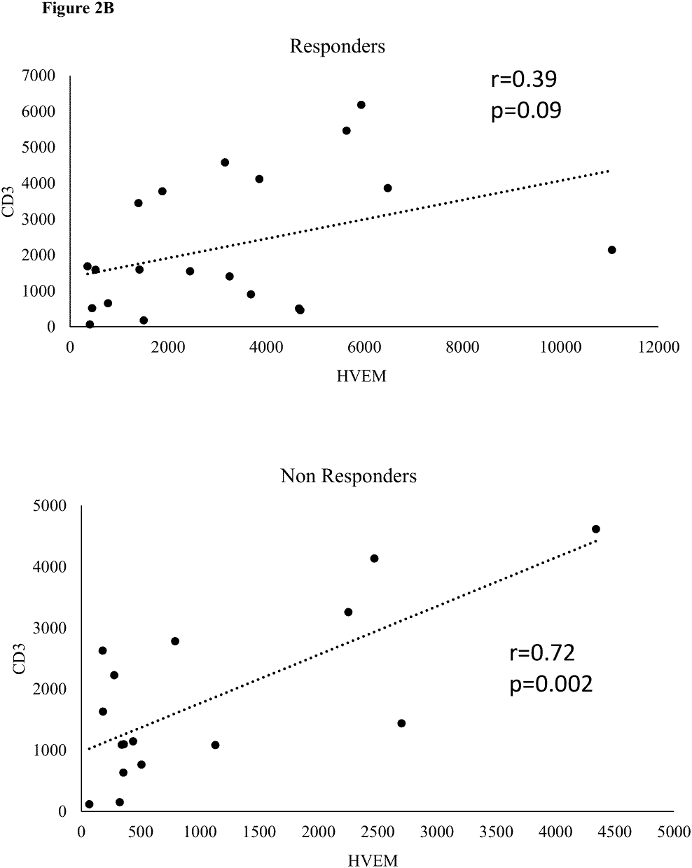

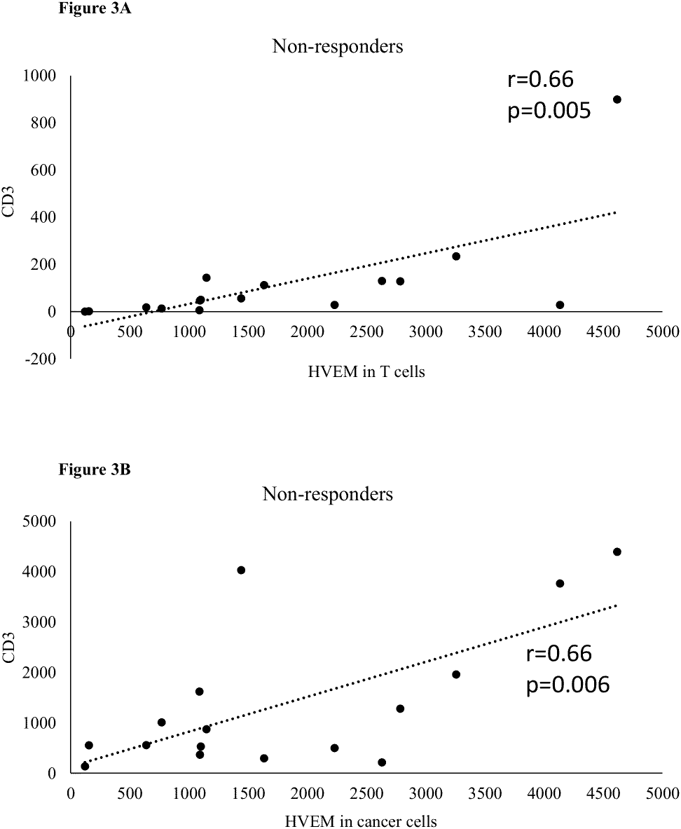

When these three markers were compared pairwise another unexpected result was observed. Comparison of CD3 expression and PD-L1 expression in the tumor samples found only a very weak correlation (responders: r=0.32, p=0.174; non-responders: r=0.32, p=0.225) which was comparable in responders and non-responders (FIG. 2A). The same weak correlation (r=0.39, p=0.09) was observed between CD3 and HVEM within the responders group (FIG. 2B, top). However, in the non-responders group a moderate to high, and statistically significant positive correlation (r=0.72, p=0.002) was observed between the expression of CD3 and HVEM (FIG. 2B, bottom). Put another way, among subjects that do not respond to anti-PD-1/PD-L1 immunotherapy, those with a high infiltration of T cells likely also have high HVEM expression. Since HVEM can be expressed both in cancer and T-cells, the correlation in each cell population was tested in the non-responders group. The correlation to HVEM expressed on T cells (FIG. 3A, r=0.66, p=0.00.5) was significant and comparable to the correlation to HVEM expressed on cancer cells, (FIG. 3B, r=0.66, p=0.006).

Combined anti-HVEM and anti-PD-1/PD-L1 therapy has been shown to be effective against PD-L1/HVEM expressing cancers (see International Patent Application WO2020222235, herein incorporated by reference in its entirety). It was additionally shown that tumors with higher HVEM expression were the most likely to respond to HVEM therapy (see Aubert et al., “TNFRSF14 (HVEM) is a novel immune checkpoint for cancer immunotherapy in humanized mice” preprint on biorxiv.org, doi.org/10.1101/711119). This new result indicates that there is another way to determine which subjects are most likely to be treatable with either HVEM monotherapy or combined HVEM/PD-1/PD-L1 therapy. Within the population of PD-1/PD-L1 non-responders T cell infiltration is indicative of HVEM level. The infiltration of immune cells into the tumor microenvironment suggests the potential to turn the tumor from an altered immunosuppressed immune tumor into a hot immune tumor, suitable to receive HVEM monotherapy or a combined treatment to improve ineffective PD-1/PD-L1 therapy.

### Example 2

TMAs of other PD-1 treatable cancers containing responder and non-responder samples are selected: including lung cancers (non-small cell lung cancer), renal cancer (renal cell carcinoma), head and neck cancer, bladder cancer and cervical cancer. Staining for HVEM is carried out and quantified as hereinabove. Higher HVEM expression is found to correlate with response to PD-1/PD-L1 therapy.

In addition to protein levels, mRNA levels of HVEM is examined. Sequencing data and/or PCR analysis from responders and non-responders is analyzed and HVEM expression is quantified. HVEM mRNA levels in the subject and in tumor samples is found to correlate with response to PD-1/PD-L1 therapy.

An analysis is also performed in which unknown samples are evaluated for HVEM expression and categorized as responsive or non-responsive. This is performed with samples from subject who have not yet been treated and the treatment outcome is recorded and found to match with the prediction. Additionally, samples which are known to be from responders/non-responders are provided as a blind sample in which those analyzing do not know the status. Once again, HVEM expression above a cutoff is indicative of responsiveness to therapy.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of determining suitability of a subject in need thereof to be treated by a PD-1/PD-L1 based immunotherapy, the method comprising receiving a sample from said subject and determining Herpesvirus entry mediator (HVEM) levels in said sample, wherein expression of HVEM above a predetermined threshold indicates the subject is suitable to be treated by said PD-1/PD-L1 based immunotherapy and treating a suitable subject with said PD-1/PD-L1 based immunotherapy, thereby determining suitability of a subject to be treated by a PD-1/PD-L1 based immunotherapy.

2. The method of claim 1, wherein said predetermined threshold is an expression level in a healthy sample or in a disease sample from a non-responder to said PD-1/PD-L1.

3. The method of claim 1, wherein said HVEM levels is HVEM mRNA levels or HVEM protein levels.

4. The method of claim 3, wherein said HVEM levels is HVEM protein levels and said determining comprises contacting said sample with an agent that binds specifically to an extracellular domain of HVEM, optionally wherein said agent is an anti-HVEM antibody comprising three heavy chain CDRs (CDR-H) and three light chain CDRs (CDR-L), wherein: CDR-H1 comprises the amino acid sequence set forth in SEQ ID NO: 1 (SYAMS), CDR-H2 comprises the amino acid sequence as set forth in SEQ ID NO: 2 (AISGSGGSTYYADSVKG), CDR-H3 comprises the amino acid sequence as set forth in SEQ ID NO: 3 (APGDYTAYFDY), CDR-L1 comprises the amino acid sequence as set forth in SEQ ID NO: 4 (RASQSVSSYLA), CDR-L2 comprises the amino acid sequence as set forth in SEQ ID NO: 5 (GASSRAT), and CDR-L3 comprises the amino acid sequence as set forth in SEQ ID NO: 6 (QQYGSSPPYT), optionally wherein said agent comprises a detectable moiety or wherein said determining further comprises contacting said sample with a secondary antibody comprising a detectable moiety.

5. (canceled)

6. (canceled)

7. The method of claim 1, wherein said sample is a disease sample, a biopsy, a bodily fluid sample, a blood sample, a serum sample or a plasma sample.

8. (canceled)

9. (canceled)

10. (canceled)

11. The method of claim 1, wherein said subject suffers from a disease or condition treatable by PD-1/PD-L1 based immunotherapy.

12. The method of claim 1, wherein said subject suffers from cancer.

13. The method of claim 12, wherein said cancer is a solid cancer.

14. The method of claim 1, wherein detecting HVEM levels comprises detecting HVEM levels on a cell surface and/or in a cytoplasm of a cell in said sample.

15. The method of claim 1, wherein detecting HVEM levels comprises detecting circulating HVEM levels.

16. The method of claim 1, wherein said PD-1/PD-L1 based immunotherapy is PD-1 and/or PD-L1 blockade.

17. (canceled)

18. A method of determining suitability of a subject non-responsive to PD-1/PD-L1 based immunotherapy to be treated with an HVEM based immunotherapy, the method comprising receiving a sample from said subject and determining T cell levels in said sample, wherein T cell levels above a predetermined threshold indicates said subject is suitable to be treated by said HVEM based immunotherapy, and treating a suitable subject with an HVEM based immunotherapy, thereby determining suitability of a subject non-responsive to PD-1/PD-L1 based immunotherapy to be treated with an HVEM based immunotherapy.

19. The method of claim 18, wherein said subject suffers from a disease or condition treatable by PD-1/PD-L1 based immunotherapy and HVEM based immunotherapy.

20. The method of claim 19, wherein said subject suffers from cancer.

21. The method of claim 20, wherein said cancer is a solid cancer or a hematological cancer.

22. The method of claim 18, wherein said sample is a disease sample, a biopsy a bodily fluid sample, a blood sample, a serum sample or a plasma sample.

23. (canceled)

24. The method of claim 18, wherein determining T cell levels comprises measuring CD3 expression in said sample, counting T cells or both.

25. (canceled)

26. The method of claim 18, wherein said T cells are tumor infiltrating lymphocytes (TILs).

27. The method of claim 18, wherein a suitable subject is suitable to receive a combined HVEM based immunotherapy and a PD-1/PD-L1 based immunotherapy and said method further comprises treating said subject with said PD-1/PD-L1 based immunotherapy in combination with said HVEM based immunotherapy.

28. The method of claim 18, wherein said PD-1/PD-L1 based immunotherapy is PD-1 and/or PD-L1 blockade, said HVEM based immunotherapy is HVEM blockade, or both.

29. (canceled)

30. (canceled)


SEQUENCE LISTING (19 sequences)

SEQ ID NO: 1 - protein, 5 aa, synthetic construct
  REGION 1..5: note = Synthetic
SYAMS

SEQ ID NO: 2 - protein, 17 aa, synthetic construct
  REGION 1..17: note = Synthetic
AISGSGGSTYYADSVKG

SEQ ID NO: 3 - protein, 11 aa, synthetic construct
  REGION 1..11: note = Synthetic
APGDYTAYFDY

SEQ ID NO: 4 - protein, 11 aa, synthetic construct
  REGION 1..11: note = Synthetic
RASQSVSSYLA

SEQ ID NO: 5 - protein, 7 aa, synthetic construct
  REGION 1..7: note = Synthetic
GASSRAT

SEQ ID NO: 6 - protein, 10 aa, synthetic construct
  REGION 1..10: note = Synthetic
QQYGSSPPYT

SEQ ID NO: 7 - protein, 120 aa, synthetic construct
  REGION 1..120: note = Synthetic
QVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKAPGDYTAYFDYWGQGTLVTVSS

SEQ ID NO: 8 - protein, 108 aa, synthetic construct
  REGION 1..108: note = Synthetic
ELVLTQSPATLSLSPGERATLSCRASQSVSSYLAWYQQKPGQAPRLLIYGASSRATGIPDRFSGSGSGTDFTLTISRLEPEDFAVYYCQQYGSSPPYTFGQGTKVEIK

SEQ ID NO: 9 - protein, 466 aa, synthetic construct
  REGION 1..466: note = Synthetic
MGWSCIILFLVATATGVHSQVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 234 aa, synthetic construct
  REGION 1..234: note = Synthetic
MGWSCIILFLVATATGVHSELVLTQSPATLSLSPGERATLSCRASQSVSSYLAWYQQKPGQAPRLLIYGASSRATGIPDRFSGSGSGTDFTLTISRLEPEDFAVYYCQQYGSSPPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 8 aa, synthetic construct
  REGION 1..8: note = Synthetic
GFTFSSYA

SEQ ID NO: 12 - protein, 8 aa, synthetic construct
  REGION 1..8: note = Synthetic
ISGSGGST

SEQ ID NO: 13 - protein, 13 aa, synthetic construct
  REGION 1..13: note = Synthetic
AKAPGDYTAYFDY

SEQ ID NO: 14 - protein, 6 aa, synthetic construct
  REGION 1..6: note = Synthetic
QSVSSY

SEQ ID NO: 15 - skipped

SEQ ID NO: 16 - protein, 10 aa, synthetic construct
  REGION 1..10: note = Synthetic
QQYGSSPPYT

SEQ ID NO: 17 - protein, 283 aa, Homo sapiens
MEPPGDWGPPPWRSTPKTDVLRLVLYLTFLGAPCYAPALPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVWWFLSGSLVIVIVCSTVGLIICVKRRKPRGDVVKVIVSVQRKRQEAEGEATVIEALQAPPDVTTVAVEETIPSFTGRSPNH

SEQ ID NO: 18 - protein, 184 aa, Homo sapiens
MEPPGDWGPPPWRSTPKTDVLRLVLYLTFLGAPCYAPALPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTK

SEQ ID NO: 19 - protein, 199 aa, Homo sapiens
MVSRPPRTPLSPSSWTPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVWWFLSGSLVIVIVCSTVGLIICVKRRKPRGDVVKVIVSVQRKRQEAEGEATVIEALQAPPDVTTVAVEETIPSFTGRSPNH